(19) United States
(12) Patent Application Publication
(10) Pub. No.: US 2007/0208278 A1
(43) Pub. Date: Sep. 6, 2007

(54) Posturo-graphic method using four three dimensionally movable platforms

(76) Inventor: Reuven Kohen-Raz, Jerusalem (IL)

Correspondence Address: R. Kohen-Raz, 11 Shmaryahu Levin Str, Jerusalem 96664, IL

(21) Appl. No.: 11/630,530

(22) PCT Filed: Jul. 5, 2004
(86) PCT No.: PCT/IL04/00596

§ 371 Date: Dec. 26, 2006

(51) Int. Cl. A61B 5/103; A61B 5/117

(52) U.S. Cl. 600/587; 600/595; 600/592

(57) ABSTRACT

A posturographic method to investigate the human postural system by calculating interactions between pressure fluctuations over time on four separate portable platforms (FIG. 1), each put beneath a subject's heel and tow part respectively. The platforms can be positioned in varying positions, as well as superimposed on a secondary surface (A-H), which in turn can be tilted, rotated, as well as simultaneously tilted and rotated generating spiral motion. Six secondary pressure interaction scores (between 2 heels and 2 toes, heel and toe of each foot and each heel and its contralateral toes) based on 4 primary parameters (Weight Percentage, Sway, Fourier Spectral Intensities and Fourier Harmony) are computed.

### TECHNICAL FIELD

Mechanical, Computerized Engineering of Medical Instruments, Diagnostic Equipment, Physical Treatment Devices

### BACKGROUND ART

The application of posturography to objectively assess disorders of human balance, which in turn are known to be related to vestibular, central nervous and orthopedic pathology is an established art in contemporary medical research and practice. (2, 3, 6) However all the hitherto constructed posturogaphic devices and methods are based on the assumption that the rather complex mechanisms of postural control can be reliably measured by analyzing postural sway manifest in displacement patterns of the center of gravity or center of pressure of the human body whilst standing on a pressure sensitive platform. Implicitly it is assumed that the human body can be bio-mechanically treated as an inverted pendulum, ankle and hip joints serving as main pivoting points. (5, 6) Hence measures of body sway are used as the main parameter stochastically expressed as a score of deviations, with limits indicating loss of balance, or else as the area of an inkblot produced by the projections of body oscillations, or as the length of the sway wave signal, the velocity of the excursions of the point of gravity etc. Furthermore the location of the point of gravity in relation to the center of the area of support is calculated. Some devices are described as “dynamic” by virtue of moving the platforms in forward-backward direction, lifting them toes up and down or letting the platform pivot around its horizontal axis and thus being “sway referenced”. (3, 6). Actually, the essential aspect of dynamic posturography is the evaluation of the subject's response and adaptation to external disturbances of his balance. (3) The equipment needed for such purpose is heavy, not movable or portable, requires harnessing and its use with children and physically handicapped persons is limited, autistic children or amputees being a case in point. In addition, controlled (yet unpublished) studies have shown, that parallel clinical examinations carried our with dynamic posturography and the present invention demonstrate a higher sensitivity of the latter.

From the point of view of weight distribution patterns, Table 1 shows paradigmatically, that a conspicuous balance disorder manifest in an extremely abnormal weight distribution, between heels and toes of both feet, which is clearly detected by the invention, will show a normal location of the center of gravity when tested with prior systems, including dynamic posturography.

TABLE 1

| | LEFT | RIGHT |
|---|---|---|
| TOES | 15% | 35% |
| HEELS | 35% | 15% |

In similar vein, all hitherto used postural bio feedback systems are based on the visualization of the displacement patterns of the center of gravity, and stimulate motor coordination in the strict sense. They do not engage higher cognitive functions, as does the invention.

None of the prior posturographic systems addresses the issue of postural diagonality, i.e. diagonal weight shifts, diagonal synergetic coordination, diagonal sway. The diagnostic power of these parameters is shown on FIG. 5. In a different context, diagonal weight shifts have been shown to be related to fatigue and predict the quality of fighter pilot performance, as well as sleep disturbances.

Furthermore, parameters of prior systems are not fit to evaluate postural rigidity, which is manifest in “above normal” stability, minimal distance of the center of pressure from the center of the support area and exaggerated diagonal sway. It has been shown by yet unpublished studies that such rigidity is a typical posturographic sign of orthopedic problems of the spine.

None of the existing posturographic systems measures the harmony of postural sway. as does the Fourier Harmony Index provided by the invention. (FIG. 4a, b).

Existing methods of balance testing as well as sophisticated oto-neurological examinations monitor only the reaction of the horizontal canals of the inner ear. The invention however is able to detect the involvement of the vertical and posterior canals, by exposing the subject to slow, non stressful spiral motion and measuring changes in balance control by means of the highly sensitive, interactive parameters of the method (See FIGS. 2, 3).

### DISCLOSURE OF INVENTION

The invention uses specially designed hardware in part disclosed in a previous patent (1) and previous publications (4) to apply a new method to investigate the human postural control system. This method focuses on neuro-physiological processes which modulate the reciprocal innervation of the lower extremities and control the vertical pressure fluctuations generated by four discrete centers of support provided by the two heels and toe parts respectively. This process of perpetually fluctuating weight distributions can be described as being an expression of synergies, synchronizations or interactions, which are produced and controlled by the central nervous system and its components, the spinal, cerebellar, sub-cortical and cortical mechanisms. Hence the monitoring of these interactions provides insight into these neuro-physiological processes, responsible for the maintenance of postural control, as well for its disturbances, within relatively short experimental time, in complete absence of postural stress as well without the necessity to perturb balance by external pulls and pushes. The brevity and friendliness of the test protocol makes the method suitable to test children from the age 3 onwards, as well as to examine extremely handicapped and anxious subjects.

The hardware consists of four separate, freely movable pediforce platforms, each bearing sensors of vertical pressure mounted in a way which prevents them from being affected by the horizontal location of the weight put on the platform's surface, including the platform corners. (FIG. 1) The four platforms can be positioned in a variety of static constellations, as well as placed on a secondary surface, which in turn can be tilted and rotated as well as simultaneously tilted AND rotated i.e. put in spiral motion. (FIGS. 2, 3)

A specially designed Control Panel which is an integral part the software offers the options to program the static positions and dynamic displacements of the platforms.
a) Static positioning: (i) Select: Platforms on the level, toes up, toes down (ii) Define degree of static tilt
b) Dynamic Tilt: (i) Define degree of maximal tilt (ii) Define speed of up-down motion.
c) Rotation: Define speed of rotation in degree/sec
d) Spiral Motion Patterns obtained by combinations of programs a) b) c) d)

By collection of data on healthy adults and children tested on various static and dynamic positions as explained above, a normative data base is established which is at the disposal of the user. The software however allows for a deliberate creation of additional data bases by the user of the system, including the ad hoc collection of comparative data. In such context advantage can be taken of the portable hardware which can be easily transferred to schools, clubs, hospitals , clinics etc to test comparison groups

The electronic signals yielded by the pressure transducers over time are digitalized and inserted into the computer via the UBS port.

The data base has 6 sections, each storing the following sectors of information: SECTION I The digitalized raw signals retrieved by the program in real time for all purposes of elaboration, comparison, graphic representation etc, as well as for the purpose of export via e-mail and internet SECTION II The personal information of the tested subject. SECTOR III The default values for the static constellation of the plates and the parameters defining static tilting and spiral motion of the secondary surface. SECTION IV The normative data based on the default format of the software SECTION V

Ad hoc values for the static constellation of the plates and ad hoc parameters defining tilting and spiral motion of the secondary surface as selected by the user SECTION VI Newly collected comparative data based on an alternative format of the software as designed by the user.

At the first level of data elaboration the program assesses the following parameters: a) Weight distribution for each plate b) Pressure fluctuation patterns c) Basic Fourier Spectrum of the wave signal d) Spectral Intensity Scores for discrete sections of sway frequencies between 0.01 to 5 Hz. e) Fourier Harmony Index which measures the deviation of the descending slope of the above Fourier Intensity Scores from a theoretical mathematical function reflecting optimal energy distribution between the above mentioned sway frequency ranges during normal stance. (FIG. 4 a, b).

At the second level of data elaboration the program computes 6 patterns of interactions between the respective parameters described above as obtained by the 6 possible combinations of paired wave signals generated on four plates. (between 2 heels and 2 toes, heel and toe of each foot and each heel and its contra-lateral toes) An extremely disturbed interaction pattern is graphically presented on FIG. 6.

At a third level of data elaboration the program will automatically compare all the measures as described above to the normative or comparative data stored in the respective sections of the data base, as described above, plot the significant deviations in graphical and numerical format etc to be inspected on spot. In addition, all outputs of two examinations of the same subject and their numerical and graphical derivatives can be displayed and plotted simultaneously, allowing for convenient comparison. The numerical data can be automatically transferred to computerspread sheets for statistical analysis.

The raw signals, as well as the elaborated data can be exported via e-mail to other users of the invention for evaluation, consultation or exchange of experience, as well transferred to computer net works.

An additional embodiment of the method is its use as screening device. The subject is instructed to maintain a simple, non stressful stance for an experimental period of short duration (50 to 75 seconds) whilst being suspect to be under the influence of drugs, fatigue, intoxication etc. (screening test) This performance is compared to a previous test response (stored in the computer) of the same subject examined with the same test given 4 times successively on a different day whilst the subject is sober, fully rested and relaxed. (base line test) The result of the comparison will be flashed by the computer in the form of a yes/no statement in respect of the existing risk status of the subject

A third embodiment of the method is its use as a postural biofeedback involving the training to avoid synkinetic movements and the stimulation of higher cognitive processes. as well the ability of spatial orientation. In this context the subject is required to reduce pressure on one of the footplates by lifting movements, whilst maintaining pressure on the rest of the plates. The such generated pressure discrepancy serves as trigger to move the cursor on the computer screen in programmed directions. This pedal maneuver can be deliberately incorporated into existing or specially designed computer games.

### APPLICABILITY

Due to the simple, non stressful and brief intake procedure on the one hand, and the rich and differentiated, output based on a set of mutually independent parameters the invention conspicuously widens the existing field of posturography as a diagnostic tool and postural biofeedback training system, as detailed below:

1) Differential diagnosis of learning disabilities and dyslexia. Prescription of remedial methods involving balance training and stimulation of spatial orientation by means of pedal interactive biofeedback

2) Monitoring drug effects and drug side effects in the context of treating vertiginous diseases and psychiatric disorders. Incorporation of the invention in legal procedures of testing new drugs before being licensed.

3) Clarification of the multi-causal etiology of factors involved in the risk of fall, especially in elderly populations. Incorporation of the invention to develop screening procedures for geriatric populations to prevent accidents. Introducing balance biofeedback as prophylactic tool in geriatric practice.

4) Accident prevention by detecting fatigue as manifest in weakened balance, by administration of posturographic tests to drivers , pilots and flight control personnel. Short obligatory screening tests to be given to workers before night shifts or before starting jobs involving postural stress, such as climbing on scaffold etc.

5) Clarification of the multi-dimensional symptoms of Whiplash injuries, their long term effects and medico-legal aspects. Posturographic screening as obligatory part of accident insurance policy

6) Early detection and treatment of diseases affecting large proportions of the contemporary world's population, such as Parkinson, Alzheimer, Vestibular Disorders, Diabetic Neuropathy, Lesions and Deformations of the spine, especially cervical and Lower Back problems, complications of pregnancy, etc In such context it is possible to propose routine posturographic screening procedures as integral part of social insurance policy and medical check up routines.

7) Early detection and prevention of pathologies caused by exposure to noxious agents during work. Obligatory balance testing for the purpose of setting up satisfactory terms of workers' insurance and indemnity claims

### REFERENCES

(1) French Patent 92-05279 Apr. 29,1992
(2) U.S. Pat. No. 5,388,591 Feb. 14, 1995
(3) U.S. Pat. No. 5,551,445 Sep. 3,1995
(4) Kohen-Raz, Application of Tetraxiametric Posturography in Clinical and Developmental Diagnosis. Perceptual & Motor Skills, 1991, 73, 635-656
(5) Kapteyn, T. S. The stabilogram. Measurement techniques. Agressologie, 1972, 13 C, 75-78.
(6) Harstall Ch. Dynamic Posturography. Alberta Heritage Foundation for Medical Research. 1998

### LIST OF FIGURES

FIG. 1 Four Plate System and Platform Constellations

FIG. 2 The Secondary Surface (Lateral View)
Legend of FIG. 2:
(A) Upper Platform bearing Footplates
(B) Tilting Body
(C) Rotating Body
(D) Lower Platform supporting Device
(E) Fixed Rod within D
(F) Spinning Wheel rotating C around axis E
(G) Wiggling Screw tilting A via B
(H) Motor inbuilt in C propelling F and G

FIG. 3 The Secondary Surface (Top View) Legend See FIG. 2

FIG. 4 Graphical Presentation of Fourier Harmony
FIG. 4a Normal Fourier Harmony
FIG. 4b Deviant Fourier Harmony in Woman with Orthopedic Spine Problem

FIG. 5 Diagonal Weight Displacement Pattern
Diagonal Weight Load on Heel+Contra-lateral Toes in Two Groups of Patients with Lateralized Back Pain
Left Figure: Back Pain on Left Side Right Figure: Back Pain on Right Side
   Black Rectangles: Left Diagonal Weight Load.
   Grey Rectangles: Right Diagonal Weight Load. Left

FIG. 6 Disturbed Sway Interaction on Four Plates in Autistic Child

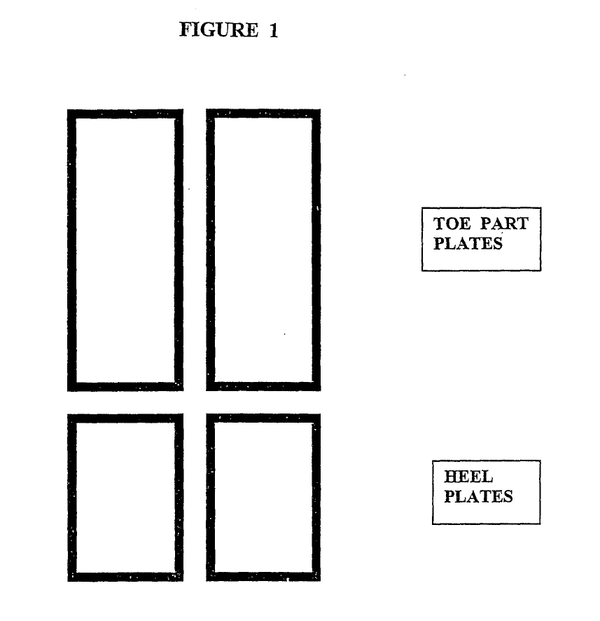

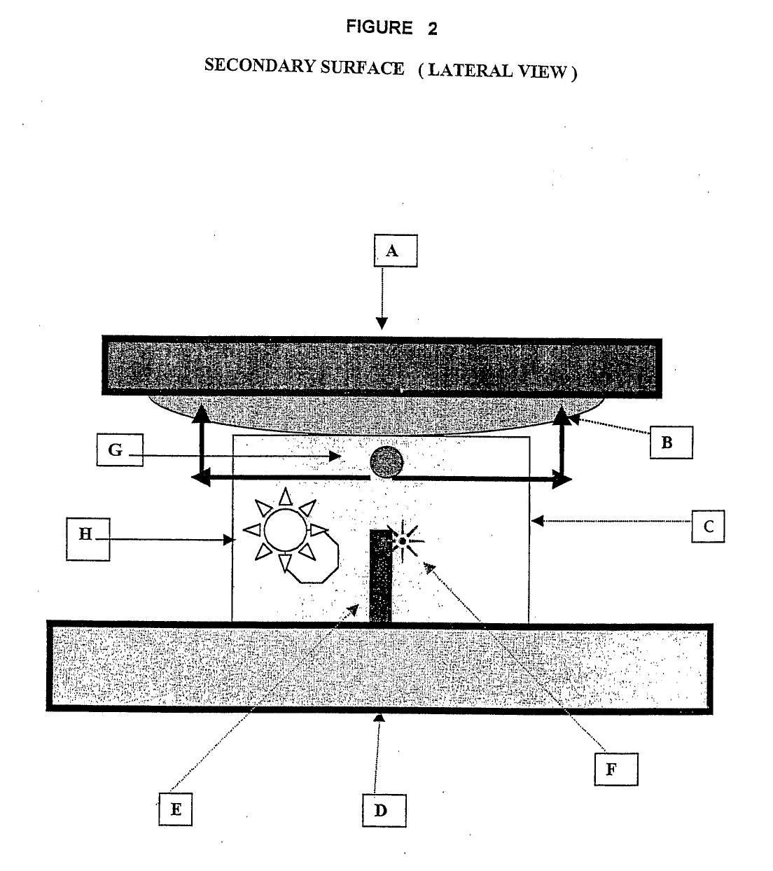

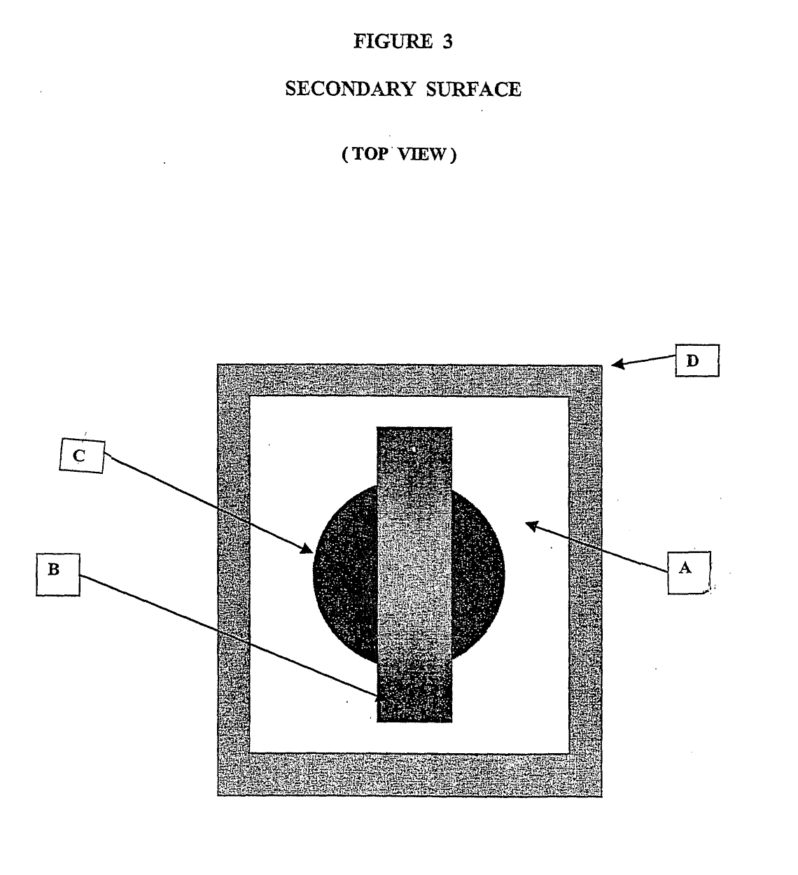

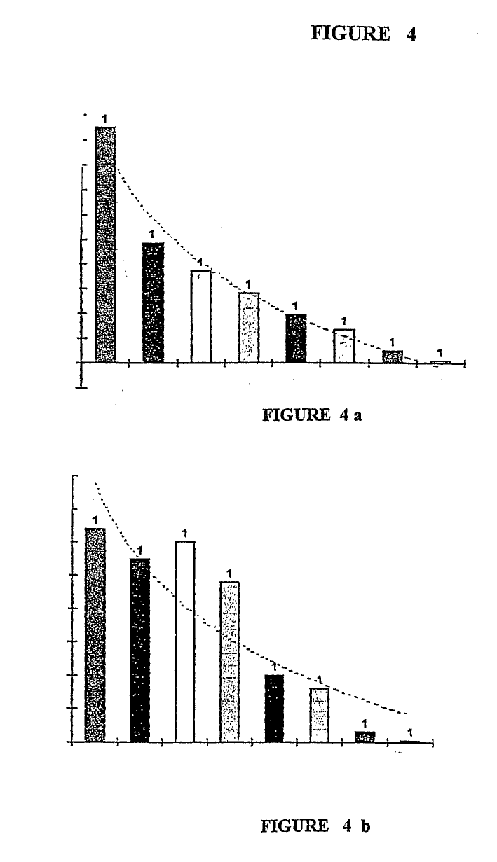

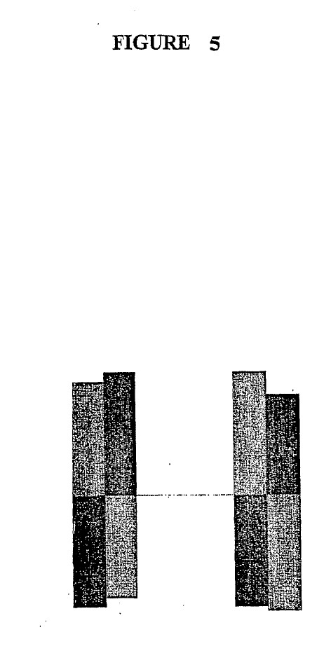

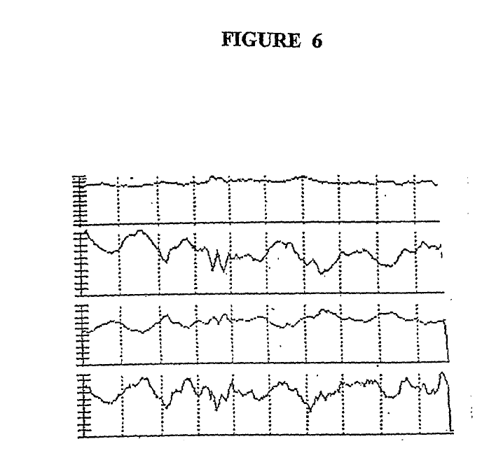

1) A method of objective assessment of human postural control based as a first step on the acquisition of four electronic wave signals emitted by at least four pressure transducers mounted each on four independent platforms, said transducers responding each separately to the fluctuating vertical forces generated by each heel and each toe-part of a person standing steady on said platforms mounted on a secondary platform which as a second step can be kept steady, as well as tilted, shifted forwards, backwards, as well as rotated (See FIG. 2), whilst as a third step the said four independent signals are digitalized by means of an analogue to digital transformer and fed via the USB port into a computer, where as a fourth step by means of a specially designed program using specially constructed algorithms a graphic and numerical output of a variety of posturographic parameters is created, reflecting the interactions and synchronizations between the said four signals taking advantage of the degrees of freedom granted by the independence of the four inputs and the additional degrees of freedom granted by the three dimensionally mobility of said secondary platform.

2) The method according to claim 1 to including the possibility to feed into the computer the personal details of the examinee.

3) The method according to claim 1 including the possibility to feed into the computer a program controlling the experimental manipulation of the secondary platform, namely a) tilting; b) linear, circular and spiral motion; c) the speed of the motions and displacements of said secondary platform.

4) The method according to claim 1 comprising in said specially designed computer program a data base, into which the posturographic data, as generated by step one, three and four of the method can be systematically stored for clinical evaluation.

5) The method according to claims 1 including in said specially designed computer program a data base, into which the posturographic data, as generated by step one, three and four of the method can be systematically stored and sorted for the purpose of creating pools of comparative data gathered on normal populations and clinical groups.

6) The method according to claims 1 and 3, including in said specially designed computer program a data base, into which the computerized protocol of the experimental manipulations of the secondary platform are stored.

7) The method according to claim 1 comprising at a first level of data elaboration the computation of a posturographic parameter measuring the amount of vertical pressure fluctuations on the four plates, serving as an indicator of general stability.

8) The method according to claim 1 comprising at a first level of data elaboration the computation of a posturographic parameter measuring the different percentages of weight put on each of the four plates, serving as indicator of the weight distribution pattern of the examined subject's body weight over the 4 plates.

9) The method according to claim 1 comprising at a first level of data elaboration the computation of a Fourier Spectral Analysis of the pressure fluctuations on each of the four plates, serving as an indicator of spectral patterns of sway intensities within eight discrete sway frequency bands extending from of 0.01 to 5.00 Hz.

10) The method according to claim 1 comprising at a first level of data elaboration the computation of a posturographic parameter expressing the correlations between the vertical pressure fluctuations as obtained on two plates selected out of the set of four plates of the system, offering six possible combinations of paired comparisons which indicate synchronizations of sway patterns between two different foot-parts, namely between the left heel and right heel, between the left toe part and the right toe part, between the left heel and the left toe part, between the right heel and the right toe part, between the left heel and right toe part and between the right heel and the left toe part.

11) The method according to claims 7, 8, 9 comprising at a second level of data elaboration the computation of correlations between the posturographic parameters of Stability, Weight Distribution, and Fourier Spectral Analysis as obtained on a single heel plate and its contra-lateral toe plate, yielding parameters of Diagonal Stability, Diagonal Weight Distribution and Diagonal Fourier Spectral Sway Patterns.

12) The method according to claims 1, 7, 8, 9, 10, 11 comprising at a third level of data elaboration the assessment of effects of tilting, shifting, and rotating the secondary platform on said parameters of Stability, Weight Distribution, Fourier Spectral Patterns, Synchronization, Diagonal Stability, Diagonal Weight Distribution and Diagonal Fourier Spectral Patterns.

13) The method according to claims 1, 5, 7, 8, 9, 10, 11, 12 comprising a program comparing the values of the parameters as obtained by the procedures described in claims 7, 8, 9, 10, 11, 12 to the values of the comparative and normative data base described in claim 5.

14) The method according to claims 1, 7, 8, 9, 10 comprising the possibility to assess by systematic research critical values of the parameters of Stability, Weight Distribution, Fourier Spectral Patterns and Synchronizations said values being indicators of high risk to loose equilibrium and orientation in space and thus implicitly predicting high risk of accidents.

15) The method according to claims 1, 7, 8, 9, 10 comprising the possibility to assess by systematic research critical values of the parameters of Stability, Weight Distribution, Fourier Spectral Patterns and Synchronizations said critical values being transformed by a computer program into yes/no signals displayed on the computer screen or being printed out, said signals indicating presence or absence of high risk to loose balance and orientation in space, said procedure serving as a quick screening method for the purpose of detecting high risk of loosing balance and orientation in space and thus implicitly serve as a method to prevent fatal accidents.

16) The method according to claim 1 comprising the possibility to develop a computer program which projects the pressure fluctuation signals obtained on four separate foot plates onto the computer screen visible to the examinee who stands on said plates, said procedure enabling the examinee to view and to control his postural sway, said computer program providing postural feedback for the purpose of training and rehabilitation of persons with postural disturbances.

17) The method according to claim 1 and claim 16 instructing the examined person to put excessive pressure on a single plate out the four plates, or alternatively removing pressure from a single plate out of the four footplates, whilst keeping moderate pressure on the remaining three plates, said maneuver producing computerized signals which operate computer games projected on the computer screen visible to the examinee standing on the all the four plates, said projection procedure being used a a postural biofeedback for the purpose of training and rehabilitation of postural and cognitive disturbances.

18) A device according to claim 1 consisting of four footplates, equipped with pressure sensitive sensors, said plates connected to a computer card transforming analogue signals into digital signals, said card connected via the USB port to a computer.

19) The device according to claim 1, said plates being mounted on a secondary platform, which by actuary means can be shifted, tilted and rotated, said shifting, tilting and rotating motion being controlled by a computer program installed on said computer. (See FIG. 2).
